(19) United States
(12) Patent Application Publication
(10) Pub. No.: US 2014/0187618 A1
(43) Pub. Date: Jul. 3, 2014

(54) COMPOSITIONS, METHODS AND KITS USING FOR INDUCING OR ENHANCING NEUROGENESIS IN CELLS AND TISSUE

(71) Applicant: Biocrescentia, LLC, Woburn, MA (US)

(72) Inventors: Milton Nascimento da Silva, Belem (BR); Alberto Cardoso Arruda, Belem (BR); Mara Silvia Pinheiro Arruda, Belem (BR); Gilmara de Nazareth Tavares Bastos, Belem (BR); Raquel Carvalho Montenegro, Belem (BR); José Luiz Martins do Nascimento, Belem (BR)

(73) Assignee: Biocrescentia, LLC, Woburn, MA (US)

(21) Appl. No.: 14/145,080

(22) Filed: Dec. 31, 2013

Related U.S. Application Data
(60) Provisional application No. 61/747,820, filed on Dec. 31, 2012.
(60) Provisional application No. 61/750,122, filed on Jan. 8, 2013.

(51) Int. Cl. A61K 31/35

(52) U.S. Cl. CPC A61K 31/35
USPC 514/450; 435/375

(57) ABSTRACT

Compositions, methods, and kits including extracts containing physalins and pure physalins are provided for inducing neurogenesis in cells and tissues. The extracts for example are obtained from natural sources such as a plant, for example Physalis angulata. The extracts and purified physalins increase proliferation of neural stem cells, differentiation of the neural progenitor cells in areas of the brain, for example the hippocampus, and are effective for treating neurodegenerative disorders, neurological disease, and neurological conditions.

### RELATED APPLICATION

The present application claims the benefit of U.S. provisional application Ser. Nos. 61/747,820 filed, Dec. 31, 2012, entitled, “Compositions, methods and kits using for inducing or enhancing neurogenesis in cells and tissue”, and 61/750,122, filed Jan. 8, 2013 entitled, “Compositions, methods and kits using for inducing or enhancing neurogenesis in cells and tissue” by inventors, Milton Nascimento da Silva, Alberto Cardoso Arruda, Mara Silvia Pinheiro Arruda, Gilmara de Nazareth Tavares Bastos, Raque Carvalho Montenegro, and José Luiz Martins do Nascimento of which each patent application is hereby incorporated by reference herein in its entirety. Further the present application is related to Brazilian patent application serial number PI1 104177-3 A2 filed Jul. 18, 2011 in the Instituto Nacional da Propriedade Industrial, entitled, “Uso da Fisalina D e dos extratos etanólico e aquoso na proliferação de células-tronco neuronais do giro denteado de camundongos adultos: Uma nova molécula neurogênica” by inventors Milton Nascimento da Silva, Alberto Cardoso Arruda, Mara Silvia Pinheiro Arruda, Gilmara de Nazareth Tavares Bastos, Raque Carvalho Montenegro, and José Luiz Martins do Nascimento.

### FIELD OF INVENTION

Compositions, methods and kits for inducing or enhancing neurogenesis in cells and tissue.

### BACKGROUND

Physalis angulata plant is popularly known as camapú, and it is found in tropical areas of Asia, Africa, and the Americas, including the Amazon rainforest. Physalis angulata has been used in traditional medicine in several countries in the form of extracts or infusions to treat various diseases such as malaria, asthma, hepatitis, dermatitis, rheumatism and gonorrhea (Lin et al. 1992 Am J Chin Med 20: pages 233-243; and Cáceres et al. 1995 J Ethnopharmacol 48: pages 85-88). The aqueous extract of the root of Physalis angulata has been observed to have analgesic effect at various doses (10 mg/kg, 20 mg/kg, 30 mg/kg, 60 mg/kg) administered intraperitoneally or orally (Bastos et al. 2006 Journal of Ethnopharmacology, v.2, n. 103, pages 241-245).

The anti-inflammatory effects of Physalis angulata were observed initially in an inflammation model of rat-paw edema induced by carrageenan (Choi et al. 2003 Journal of Ethnopharmacology, v.89, n. 1, pages 171-175), in a model of arthritis induced by formaldehyde and in an allergy model with anti-allergic activity against type IV hypersensitivity reaction induced by contact with 2.4 dinitrofluorobenzene (DNFB). A methanol extraction of flowers of Physalis angulata were orally administered in a single dose of 200 mg/kg. An anti-inflammatory effect was observed in adult mice of the aqueous extract of the roots of the same plant at different doses (0.5 mg/kg, 1 mg/kg, and 5 mg/kg) by intraperitoneal administration and using a model system (Bastos et al. 2008 Journal of Ethnopharmacology, v. 118, pages 246-251). The effects observed were a decrease in exudate volume, a decrease in the total number of cells and in nitric oxide levels, and inhibition of PGE2 (Bastos et al. 2008 Journal of Ethnopharmacology, v. 118, pages 246-251).

Physalis angulata plant has also been studied for its immunomodulating activity (Soares et al. 2003 European Journal of Pharmacology, v. 459, n. 1, pages 107-112). Purified Physalin B, Physalin F and Physalin G isolated from the ethanol extract of Physalis angulata were observed to inhibit macrophages activation by lipopolysaccharide and interferon-γ, in a cultured in vitro model. The physalins induced a substantial reduction in nitric oxide production. Physalin B was observed to significantly reduce levels of TNF-α, interleukin-6 and interleukin-12 in serum of mice treated with lipopolysaccharide.

The anti-leishmanial properties of Physalin B and Physalin F have been analyzed, and both physalins have been observed to inhibit the growth of Leishmania amazonensis in macrophages infected with the parasite (U.S. patent application Ser. No. 09/417,779, entitled “Process for isolating physalins from plants and pharmaceutical compositions containing physalins”, published Aug. 1, 2002 as U.S. application number 2002/0103386; and Brazilian patent application number umber PI0404635-8, entitled “Processo para a obtenção de esteróides seco derivados de ergostano”, each of which is hereby incorporated by reference herein in its entirety).

There is a need for new classes of molecules that have a direct functional impact on cognitive and other neuronal-associated processes and that induce neurogenesis in cells and tissue.

### SUMMARY

An aspect of the invention provides a method for inducing neurogenesis (FIG. 1) in cells or tissue, the method including: administering to the cells or the tissue an agent including a physalin, thereby inducing the neurogenesis in the cells and the tissue.

In various embodiments, administering is performed in vivo in a subject. For example, the subject is a human, a rodent, a cow, a pig, a dog, or a cat. Alternatively, administering is performed in vitro. In a related embodiment, the method involves prior to administering, culturing the cells or the tissue. For example, culturing the cells or the tissue includes using frozen cells, freshly thawed cells, or freshly collected cells.

In various embodiments of the method, the cells or the tissue include stem cells, progenitor cells, or adult cells. In various embodiments, the cells or the tissue include neural stem cells, neural progenitor cells, or adult neural cells such as astrocytes, oligodentrocytes, and neurons.

In various embodiments of the method, the subject is at risk for a neurological disease, neurological condition, or a neurological disorder. Alternatively, the subject currently has for a neurological disease, neurological condition, or a neurological disorder. For example, the neurological disease, neurological condition, or the neurological disorder is selected from the group of: aphasia; encephalomyelitis, adrenoleukodystrophy, agenesis, agnosia. Alexander disease, Alpers' disease, hemiplegia, Alzheimer's disease, and Parkinson's disease. Without being limited by any particular theory or mechanism of action, it is here envisioned that the method described herein is effective to treat any neurological disease, neurological condition, or neurological disorder that involves injury or dysfunction of neural cells or neural tissues.

In various embodiments of the method, the physalin is at least one selected from: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof. In a related embodiment, the physalin comprises at least two physalins. In a related embodiment, the at least one of the two physalins is Physalin D.

In various embodiments, the method further includes prior to administering, obtaining the physalin from a natural source. For example, the natural source is from a plant or material found in Asia, Africa, North America, or South America. In a related embodiment, the plant is from a Solanaceae family. For example, the plant is a Nicotiana or a Physalis.

In various embodiments, the physalin is synthetic or semi-synthetic. For example, the method involves obtaining a physalin from a natural source and then synthesizing the semi-synthetic physalin. For example, synthesizing involves a substitution, deletion, or modification of at least one functional group or carbon atom on the physalin from the natural source.

In various embodiments of the method, the natural source is a plant or portion thereof selected from at least one of: a leaf, a stem, a flower, a peel, a flower bud, and a fruit. For example, the portion of the plant is an aerial portion of the plant. Alternatively, the portion of the plant is a root.

In various embodiments of the method, the plant is a Physalis plant for example Physalis angulata, Physalis alkekengi, Physalis peruviana, or Physalis philadelphica.

In a related embodiment of the method, the physalin is from an extract. For example, the extract is an aqueous extract, an ethanol extract, or an organic extract. In a related embodiment of the method, obtaining the physalin includes extracting the plant with at least one fluid or solvent, for example an organic solvent. In various embodiments of the method, the at least one fluid or the solvent is selected from the group consisting of: chloroform, dichloromethane, ethyl acetate, diethyl ether, acetic acid, hexane, toluene, ethanol, acetone, methanol, tetrahydrofuran, dimethyl sulfoxide, acetonitrile, water, and a combination thereof. In various embodiments, extracting includes using at least one mixture of different fluids or solvents. In a related embodiment, obtaining the extract involves using at least one chromatographic technique. For example, the chromatographic technique involves a separation method involving at least one of: thin-layer chromatography, high pressure liquid chromatography, gas chromatography, and silica gel chromatography. In various embodiments, the separation involves collecting a plurality of elutions.

In various embodiments of the method, obtaining includes drying or lyophilizing a residue containing the physalin. For example, the drying or lyophilizing involves using an apparatus such as an evaporator.

The physalin in various embodiments of the method is synthetic and prior to administering, the method further includes synthesizing the physalin.

In a related embodiment of the method, the physalin shares at least about 50-90% homology to the following steroid structure:

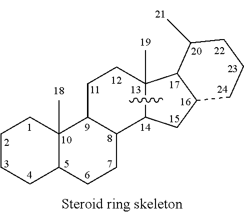

In various embodiments, the physalin includes a lactone functionality or an alcohol functionality attached to at least one of carbons 13, 14, 15, 16, 17, 20, 21, 22, 23 and 24. In various embodiments of the method, the physalin includes at least one functional group at a carbon selected from: a (C₁-C₁₈)alkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl.

In various embodiments provided herein, the physalin includes at least one functional group at a carbon selected from: a (C₁-C₁₈)alcohol, a (C₁-C₁₈)nitrile, a (C₁-C₁₈)carbonyl, a (C₁-C₁₈)ester, a (C₁-C₁₈)lactone, a (C₁-C₁₈)ether, and a (C₁-C₁₈)alkoxy.

In various embodiments of the method, the physalin includes a 13,14-seco-16,24-cyclo-steroid structure. In various embodiments, the physalin is synthesized from a steroid, such as a withanolide.

In various embodiments, prior to administering, the method includes preparing a transplant containing the cells or the tissue of the subject, such that the cells or the tissue is treated in vitro. In various embodiments, the cells or tissue contain at least one of: stem cells, progenitor cells, or adult neural cells. Alternatively, the method includes preparing a transplant containing the cells or the tissue from a transplant donor, who is not the subject. In various embodiments, preparing the transplant from the transplant donor involves matching blood type and/or antigen matching. For example, preparing the transplant includes minimizing antigenic differences between donor and recipient of the transplant by matching Human Leukocyte Antigens (HLA). HLA are also commonly known as Major Histocompatibility (MHC) antigens. In various embodiments minimizing antigenic differences between the donor and the recipient involves treating the transplant recipient with an immunosuppression agent.

In various embodiments of the method, the transplant is at least one selected from the group of: an autograft, an allograft, and a xenograft. In various embodiments, the method further includes applying an autologous or allogeneic transplant containing the cells, for example stem cells or progenitor cells, to an area of neural tissue damage or an area requiring neural tissue growth, under conditions suitable for differentiating the cells into the type of neural tissue necessary for repair. In various embodiments of the method, the particular tissue environment will be sufficient to cause proliferation and/or differentiation of the cells into their desired differentiated form. Alternatively, applying the transplant includes administering an agent that induces proliferation and/or differentiation of the cells.

The method in various embodiments further includes observing a remediation of a neurological disease or a neurological condition. For example, observing the remediation involves determining a presence of or measuring an amount or level of a marker from a sample of the subject. For example, the marker is a peptide, a protein, a carbohydrate, a sugar, a compound, or a genetic material. For example, the genetic material comprises DNA or RNA. In various embodiments, the protein comprises an enzyme. In various embodiments, the marker is a growth factor, for example a nerve growth factor.

In various embodiments of the method, administering the physalin involves administering an additional therapeutic agent. For example, the therapeutic agent comprises at least one of: a growth factor, an anti-inflammatory agent, a vasopressor agent, a collagenase inhibitor, a steroid, a matrix metalloproteinase inhibitor, an ascorbate, an angiotensin, a calreticulin, a tetracycline, a fibronectin, a collagen, a thrombospondin, an anti-viral, an anti-cancer, an anti-seizure, and an anti-coagulant. In various embodiments, the therapeutic agent is a growth factor, for example a nerve growth factor.

In various embodiments, the therapeutic agent is a protein and the method includes administering a gene carrying a vector that encodes the therapeutic agent. In related embodiments of the method, the vector is a viral vector or a plasmid for example, the viral vector is derived from a genetically engineered genome of at least one virus selected from the group consisting of adenovirus, adeno-associated virus, a herpesvirus, and a lentivirus. For example, the lentivirus is a retrovirus.

In various embodiments of the method, the therapeutic agent is selected from at least one of: a drug, a polymer, a protein, a peptide, a carbohydrate, a low molecular weight compound, an oligonucleotide, a polynucleotide, and a genetic material such as DNA or RNA.

In various embodiments of the method, administering includes at least one selected from the group of: invitreally, subcutaneously, orally, rectally, parenterally, intracisternally, intravaginally, intraperitoneally, bucally, nasally, and intra-cranially.

An aspect of the invention provides a method for making an agent that induces or enhances neurogenesis in cells or tissue, the method including: extracting a physalin from a natural plant source and administering the physalin to a subject in need of neurogenesis, wherein the physalin induces or enhances the neurogenesis.

In various embodiments of the method, the natural plant source is a plant or portion thereof selected from at least one of: a leaf, a stem, a flower, a flower bud, a seed and a fruit. In a related embodiment, the natural plant source includes an aerial portion of the plant. Alternatively, the natural plant source includes a root of the plant. In an embodiment of the method, the plant is a member of the genus Physalis.

In various embodiments of the method, extracting the physalin involves contacting the plant with at least one fluid or organic solvent, for example the fluid is an aqueous fluid. In various embodiments, extracting the physalin involves contacting the plant with ethanol or hexane. In various embodiments of the method, the at least one fluid or the solvent is selected from the group consisting of: chloroform, dichloromethane, ethyl acetate, diethyl ether, acetic acid, hexane, toluene, ethanol, acetone, methanol, tetrahydrofuran, dimethyl sulfoxide, acetonitrile, water, and a combination thereof. In various embodiments, extracting involves using a mixture or a combination of two or more fluids or solvents.

After extracting, the method in various embodiments further involves drying or lyophilizing the fluid or the solvent to obtain a residue containing the physalin.

An aspect of the invention provides a pharmaceutical composition for inducing or enhancing neurogenesis, the composition including a physalin having a 13,14-seco-16,24-cyclo-steroid structure, and a pharmaceutically acceptable diluent or carrier.

The physalin in various embodiments of the pharmaceutical composition is selected from at least one: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof. In certain embodiments, the physalin is a Physalin D with the following structure:

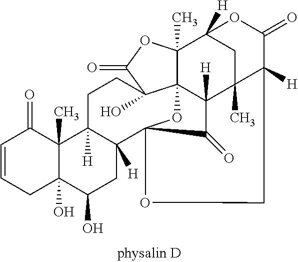

The physalin in various embodiments of the pharmaceutical composition is synthetic or semi-synthetic. For example, the physalin is a naturally-occurring physalin that is chemically-modified to produce the semi-synthetic physalin. Alternatively, the physalin is naturally occurring. In a related embodiment of the pharmaceutical composition, the naturally-occurring physalin is a plant extract. For example, the naturally-occurring physalin is an aqueous extract, an ethanol extract, a methanol extract, a hexane extract, or a chloroform extract. In various embodiments of the pharmaceutical composition, the naturally-occurring physalin is an organic solvent extract.

The physalin in various embodiments of the pharmaceutical composition has a structure having at least one a lactone functional group attached to the steroid structure of the physalin. In various embodiments of the pharmaceutical composition, the physalin has a structure having at least one alcohol functional group attached to the steroid structure of the physalin.

In various embodiments of the pharmaceutical composition, a functional group is attached to at least one of carbon atom of the steroid structure of the physalin. For example, the functional group is at one selected from: a (C₁-C₁₈)alkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl. In various embodiments of the pharmaceutical composition, the functional group is at one selected from: a (C₁-C₁₈)alcohol, a (C₁-C₁₈)nitrile, a (C₁-C₁₈)carbonyl, a (C₁-C₁₈)ester, a (C₁-C₁₈)lactone, a (C₁-C₁₈)ether, and a (C₁-C₁₈)alkoxy. For example, the functional group is attached to at least one carbon atom in a steroid structure at number: 13, 14, 15, 16, 17, 20, 21, 22, 23 and 24. In various embodiments, the pharmaceutical composition is non-toxic and the physalin effectively passes the cross the blood-brain barrier. In various embodiments, the pharmaceutical composition is formulated for oral administration or parenteral administration. In various embodiments, the pharmaceutical composition is formulated in a therapeutically effective dose for administration to a subject.

An aspect of the invention provides a kit for inducing or enhancing neurogenesis, the kit containing: a physalin including a 13,14-seco-16,24-cyclo-steroid structure and a pharmaceutically acceptable diluent or carrier; instructions for use in a subject in need of neurogenesis: and, a container.

The physalin in various embodiments of the kit has at least one selected from: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof.

In various embodiments of the kit, the physalin is naturally occurring. Alternatively, the physalin is synthetic or semi-synthetic.

In various embodiments of the kit, the physalin is naturally occurring and is from an extract of a plant. For example, the physalin is from an aqueous extract or an organic solvent extract such as an ethanol extract.

In various embodiments of the kit, the physalin includes a structure having a lactone. In various embodiments of the kit, the physalin includes a structure having an alcohol. For example, the physalin includes a diol functional group.

In various embodiments of the kit, the physalin includes at least one functional group that is attached to at least one of carbon atom of the steroid structure. For example, the physalin includes the following steroid structure:

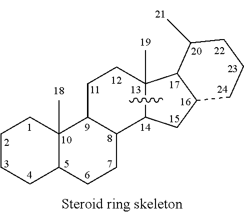

In various embodiments of the kit, the functional group is attached to at least one carbon atom in the steroid structure at number: 13, 14, 15, 16, 17, 20, 21, 22, 23 and 24.

In various embodiments of the kit, the functional group is at one selected from: a (C₁-C₁₈)alkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl. In various embodiments of the kit, the functional group is at one selected from: a (C₁-C₁₈)alcohol, a (C₁-C₁₈)nitrile, a (C₁-C₁₈)carbonyl, a (C₁-C₁₈)ester, a (C₁-C₁₈)lactone, a (C₁-C₁₈)ether, and a (C₁-C₁₈)alkoxy.

In various embodiments, the instructions for use include a method for administering the physalin to cells or tissue of the subject. For example, the instructions for use include a range of doses that are therapeutically effective.

In various embodiments of the kit, the instructions for use include a method for administering preparing a transplant of the subject and contacting the physalin to cells or tissue of the transplant. In various embodiments of the kit, the instructions for use include a method for administering preparing a transplant that is an allograft or is a xenograft. The instructions for use optionally further comprising a method for contacting the physalin to cells or tissue of the transplant. In various embodiments, the transplant includes stem cells or progenitor cells.

The kit further includes in various embodiments an applicator for contacting the physalin to the subject. For example, the applicator is selected from: a syringe, a brush, a swab, a gauze, a sprayer, a sponge, a dropper, a patch, and a pad.

A pharmaceutical composition for inducing or enhancing neurogenesis, the composition including at least one physalin selected from Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof; and a pharmaceutically acceptable diluent or carrier, such that the physalin is selected for neurogenesis activity and brain physiological tolerance at dosages effective for stimulating brain cell number.

### BRIEF DESCRIPTION OF THE DRAWINGS

FIG. 1 panels A and B are a set of drawings showing the process of neurogenesis in the dentate gyrus region of the hippocampus. Stem cells in the dentate gyrus proliferate into progenitors cells, which then differentiate into oligodentrocytes, astrocytes, or neurons. Over a period of time each of the oligodentrocytes, astrocytes, or neurons either survive or due.

FIG. 1 panel A is a drawing of the neurogenesis process that neural stem cells (first row) undertake in the dentate gyrus portion of the hippocampus. Stem cells and lineage restricted progenitor cells (second row) proliferate, and the progenitor cells differentiate into either oligodentrocyte cells (left third row); astrocyte cells (middle third row), and neural cells (right third row). These cells either undergo cellular death (fourth row) or survive (fourth and fifth row).

FIG. 1 panel B (top) is a photograph of a section of a hippocampus from a BALB/C mouse. The photograph of the hippocampus section shows the hippocampal cornu ammonis (Ammon's horn; CA) 1 area (CA1), the hippocampal CA2 area (CA2) and dentate gyrus (DG). The dentate gyrus consists of three layers of neurons: molecular, granular, and polymorphic. FIG. 1 panel B (bottom) is a drawing of a portion of the granular layer of neurons of the dentate gyrus shown in FIG. 1 panel B (top). The drawing shows that each of the neural stem cells and progenitor cells (left) proliferate. The progenitor cells differentiate into either neural cells or astrocyte cells, which either undergo cellular death or survive.

FIG. 2 panels A, B, and C showing that an aqueous extract of Physalis angulata aerial plant material, known as SM2, increased bromodeoxyuridine staining and Hoechst staining in neural cells in the subgranular zone of the dentate gyrus of six to eight week old adult BALB/C subjects. Bromodeoxyuridine dyes detect proliferating cells in living tissues. Hoechst dyes specifically bind to cells having a nucleotide sequences having adenine and thymine. Data show aqueous extract SM2 from Physalis angulata increased neural cell proliferation in the hippocampus both 24 hours and seven days after injection of SM2. The proliferation of hippocampal stem cells by the aqueous SM2 extract commenced during the period of cell proliferation and continued until the period of differentiation of the neural stem cells and neural progenitor cells into adult neural cells.

FIG. 2 panel A is a drawing of the SM2 injection schedule and the tissue collection performed on the subjects. Subjects were injected with different amounts of the aqueous SM2 extract. Control subjects were injected with saline. Subjects were then administered bromodeoxyuridine (BrdU; 50 milligrams per kilogram of weight; mg/kg) five hours later. Subjects were sacrificed and tissues were perfused with saline, were stained, and were visualized twenty-four hours later and seven days later with either BrdU dyes or Hoechst dyes.

FIG. 2 panel B is a set of photomicrographs showing BrdU staining (left most column) and Hoechst staining (second column from the left) of the subgranular zone of the dentate gyrus of BALB/C mice twenty four hours after BrdU administration and injection 0.1 milligram/kilogram body weight (mg/kg) of aqueous extract SM2 from Physalis angulata (second row), or 1 mg/kg of aqueous extract SM2 from Physalis angulata (third row). Control subjects were injected with BrdU and saline (first row). The third column photographs are merged images of the BrdU staining photographs (left column) and Hoechst staining photographs (second column from left) respectively. The fourth column is a higher resolution (Hight) image of the outlined portions in the merged images (third column). Images show enhanced BrdU staining and Hoechst staining for each of the subgranular zone of the dentate gyrus of subjects injected with aqueous extract SM2 from Physalis angulata compared to control subjects injected with saline.

FIG. 2 panel C is a set of photomicrographs showing BrdU staining of the subgranular zone of the dentate gyrus of BALB/C mice seven days hours after BrdU administration and injection with 5 mg/kg of aqueous extract SM2 from Physalis angulata (right column), or saline (control, left column). At seven days, data show that aqueous extract SM2 increased BrdU staining in cells in the subgranular zone of the dentate gyrus of SM2-injected subjects compared to BrdU staining in cells from control subjects.

FIG. 3 is a bar graph showing percent BrdU (% of BrdU⁺; ordinate) in neural cells in the subgranular zone of the dentate gyrus of the hippocampus of subjects injected with 5 mg/kg of aqueous extract SM2 from Physalis angulata (right bar) on the abscissa and administered seven days earlier with BrdU. Control subjects were injected with saline only and administered BrdU (left bar). This data show that aqueous extract SM2 increased the percentage and amount of BrdU in neural cells in the subgranular zone of the dentate gyrus of the hippocampus of SM2-injected subjects compared to cells from control subjects. Symbols used: hilo=hilus; cg=granular layer; cm=molecular layer.

FIG. 4 is a bar graph showing percent BrdU (% of cells BrdU⁺; ordinate) in neural cells in the subgranular zone of the dentate gyrus of the hippocampus of subjects injected with either 0.1 mg/kg (middle bar) or 1 mg/kg (right bar) of aqueous extract SM2 from Physalis angulata on the abscissa and administered twenty four hours earlier with BrdU. Control subjects were injected with saline only and administered BrdU (left bar). This data show that aqueous extract SM2 increased in a dose-dependent matter the percentage and amount of BrdU in neural cells in the subgranular zone of the dentate gyrus of the hippocampus of SM2-injected subjects compared to cells from control subjects.

FIG. 5 is a chart showing percent BrdU (percentage de BrdU⁺) in neural stem cells from BALB/C subject injected with either 0.1 mg/kg (middle column) or 1 mg/kg (right column) of aqueous extract SM2 from Physalis angulata and administered BrdU. Control subjects were injected saline only and administered BrdU (left column). The neural stem cells injected were obtained from the subgranular layer (first row), the granular layer (second row), or molecular layer (third row) of the dentate gyrus of the hippocampus. Aqueous extract SM2 from Physalis angulata increased in a dose-dependent matter the percentage and amount of BrdU in neural cells in the subgranular zone of the dentate gyrus of the hippocampus of subjects compared to cells from control subjects. Aqueous extract SM2 increased amount of cellular proliferation of neural stem cells of the dentate gyrus region of the hippocampus. Aqueous extract SM2 did not promote ectopic distribution of BrdU-positive cells in the hippocampus of the SM2-injected subjects. The aqueous SM2 promoted neural cell proliferation in the neurogenic zone of the hippocampus.

FIG. 6 is a bar graph showing percent of cells incorporating BrdU (% of BrdU⁺; ordinate) in neural cells in the subgranular zone of the dentate gyms of the hippocampus of subjects injected with 5 mg/kg of aqueous extract (AE) of Physalis angulata (right bar) on the abscissa and BrdU each administered every seven days for 90 days. Control subjects were injected with saline only (no AE) and BrdU (left bar) each administered every seven days for 90 days. The subjects were sacrificed on day 90 and cell uptake of BrdU was analyzed. These data show that AE of Physalis angulata causes increased the amount of BrdU and percentage uptake into neural cells in the subgranular zone of the dentate gyrus of the hippocampus of Physalis angulata AE-injected subjects compared to cells from control subjects.

### DETAILED DESCRIPTION

Neurologic diseases are disorders of any of the brain, spinal cord and nerves that can affect the entire body. Incidence of these diseases on the central nervous system and peripheral nervous system cause dysfunction in subjects including for example difficulties in movement, speech, swallowing, breathing, learning, memory, senses (e.g., touch and taste), and mood. There are more than 600 neurologic diseases or neurological conditions including: diseases caused by faulty genes such as Huntington's disease and muscular dystrophy; problems with the way the nervous system develops such as spina bifida; degenerative diseases that cause nerve cells to be damaged or die such as Parkinson's disease and Alzheimer's disease; diseases of the blood vessels that supply the brain such as stroke; injuries to the spinal cord and brain; seizure disorders such as epilepsy; infections such as meningitis; and even brain tumors. Alzheimer's disease (AD) is the most common cause of dementia among older people, affecting as many as 5.1 million Americans, and is irreversible, progressive, and destroys memory and cognitive skills, and eventually causes death.

Neurogenesis is a process by which multipotent neural stem cells (NSCs) exhibit the capacity for self-renewal and differentiation in major cellular phenotypes of the central nervous system (CNS), such as neurons, astrocytes and oligodendrocytes (Gage 2000 Science. 287:1433-1438). NSCs can be isolated from many adult tissues of the body including for example the nervous system and the skin (see Crawford et al. international application number WO/2008/137115 published May 5, 2008). NSCs in physiological conditions are located primarily in the subventricular zone of the olfactory bulb (Lois et al. 1993 Proc Natl Acad Sci USA 90: pages 2074-2077) and the subgranular zone of the hippocampal dentate gyrus (Zhao et al. 2008 Cell 132: pages 645-660).

The microenvironment of the subgranular zone (SGZ) and subventricular zone (SVZ) provide conditions that allow differentiation and integration of new neurons (Zhao et al. 2008 Cell 132: pages 645-660). Researchers have shown presence of neural stem cells in adult mammalian brains in the subventricular zone and the hippocampal dentate gyrus (Gage et al. 2002 Journal of Neuroscience Research 69 (6): pages 745-749). The neural stem cells in these areas of the hippocampus exhibited the ability to proliferate and differentiate into neurons and glial cells (Ono et al. 2001 Dev Biol 231: pages 77-86). Following maturation, the neural cells integrated and formed neural connections.

Neurogenesis in the hippocampus is complex process that occurs in the dentate gyrus and begins with proliferation of NSCs in the SGZ, followed by cell differentiation and spontaneous migration into the granular layer, and finally maturation with possible survival or neural cell death (Eriksson et al. 1998 Nature Medicine (4): pages 1313-1317). In the dentate gyrus of rodents, newly formed neuronal cells in the SGZ migrate into the granule cell layer, and differentiate into neuronal cells and increase axonal projections to the CA3 hippocampal area (Altman et al. 1965 J Comp Neurol 124: pages 319-335.). In rats, immature granule cells extend their axons into the CA3 between four to ten days after the onset of mitosis. The maturation of the granule cells that formed and proliferate in the SGZ, their migration to the granule layer, and their differentiation into neuronal cells takes roughly four weeks (Cameron et al. 1993 Neuroscience 56:337-344; and Hastings et al. 1999 J Comp Neurol. v. 413 pages 146-154).

A quantitative analysis of the neurons newly generated in rodent brains determined that approximately nine thousand new neurons were generated, such that an increase of 3.3% per month in the population of subgranule cells in the dentate gyrus was observed (Kerpermann et al. 1997 Nature 386: pages 493-495.). Researchers found that in the brains of adult primates, it is estimated that at least 0.004% of the neuronal population in the subgranule cell layer are generated each day (Kornack et al. 1999 Proc Natl Acad Sci USA 96: pages 5768-5773). Therefore, the relative rate of neurogenesis in adult primates is ten-fold less than in the dentate gyrus of adult rodents (Ibid.). Neurogenesis in the dentate gyrus (DG) of adult humans was analyzed by Bromodeoxyuridine (BrdU) immunofluorescence staining in brain tissue samples obtained from patients who died of cancer. The BrdU technique was used to assess the proliferative activity of hippocampal cells and strong staining was observed in slices of dentate gyrus from the patients. However, this study did not perform a quantitative study that could estimate the percentage of neurons generated (Eriksson et al. 1998 Nature Medicine (4): pages 1313-1317).

In the SGZ, the hippocampal progenitors are located opposite to the granule cell layer that includes immature and mature neurons, and within this SGZ microenvironment, there are also astrocytes, oligodendrocytes and other types of neurons (Zhao et al. 2008 Cell 132: pages 645-660). Hippocampal astrocytes play a critical role in neurogenesis in the SGZ by promoting the differentiation of neural progenitors and integration of these new neurons in vitro in the hippocampi of adult rats (Song et al. 2002 Nat Neurosci 5: 438-445). Furthermore, neurogenesis in adults is influenced and regulated by various physiological and pathological factors, such as emotional state, and psychological and pathological changes (Christie et al. 2006 Hippocampus. 2006; 16(3): 199-207; and Elder et al., 2006 Mt Sinai J. Med. 73(7):931-40). Models of neuroinflammation induced by peripheral administration of bacterial endotoxin lipopolysaccharide (LPS) in adult mice were used and researchers detected an intense activation of microglial cells, which release inflammatory cytokines and promote the reduction of neurogenesis in the hippocampal dentate gyrus. The microglial cells inhibited the survival of stem cells (Monje et al. 2003 Science. 2003; 302 (5651): pages 1760-1765; and Bastos et al. 2008 Neuroscience 155: pages 454-462).

Neurogenesis can also be influenced by the mood of the individual: in conditions such as depression, there is a reduction in the number of proliferating cells in the subgranule zone of the dentate gyrus. An increase in the level of corticosteroids decreased cell division in the dentate gyrus of adult rats. Corticosteroid levels are often elevated in animals experiencing stress and in patients with depression (Gould et al. 1993 J Neurosci 12: pages 3642-3650). Morphological changes have also been demonstrated in the hippocampus in response to stress. The morphological changes observed after exposure to physical or psychological stress include atrophy and loss of CA3 pyramidal neurons (McEwen 1999 Annu Rev Neurosci 22: pages 105-122).

The use of antidepressants, including fluoxetine, reverses or prevents the reduction of stress-induced neurogenesis (Saxe et al. 2006 Proc Natl Acad Sci USA. 103(46): pages 17501-17506; Encinas et al. 2006 Proc Natl Acad Sci USA. 103(21): pages 8233-8238; Malberg et al. 2000 J. Neurosci. 20(24): pages 9104-10). Thus, it is possible that modulation of the proliferation, migration and differentiation of neural stem cells is a very promising strategy for maintaining plasticity and preserving memory capacity in patients with senile memory deficits. Additionally, there is progressive neuronal death and reduction in the regenerative capacity of the brain in neurodegenerative diseases such as Alzheimer's and Parkinson's, which may be due to the reduction in cell proliferation and formation of new neurons in the dentate gyrus (Lindvall et al. 2006 Nature 441(7097):1094-6).

There is a need for new drugs or substances that enhance cognition and other neuronal-associated processes, and that influence and expand brain activities such as learning and memory. These drugs and substances may be of natural origin, or at least similar to natural substances found endogenously, and should not be illicit or trigger chemical dependency. Thus, there is a need for new classes of molecules that have a direct functional impact on cognitive and neuronal-associated processes. Without being limited by any particular theory or mechanism of action, it is here envisioned that Physalis extracts, such as aqueous extracts (AE), ethanol extracts (EE) of Physalis plants, and pure physalins such as Physalin D are effective neurogenesis promoters and neurogenesis agents.

Methods for isolating an aqueous extract (AE), an ethanol extract (BE), and a pure Physalin D preparation from Physalis angulata are shown herein. Examples herein performed a systematic in vitro and in vivo analysis of the neurogenic effects of physalins and extracts from Physalis angulata that contain physalins. Data herein show that a substantial neurogenic effect of Physalis angulata extracts and pure physalins on hippocampal stem cells of adult subjects. The extracts of Physalis angulata and pure physalin molecules were effective for proliferating neural cells in the hippocampal neurogenic niche of subjects.

Physalins are steroidal constituents of Physalis plants which possess a characteristic 13,14-seco-16,24-cyclo-steroidal ring skeleton in which the bond that is normally present between the 13 and 14 positions in other steroids is absent, and a new bond between positions 16 and 24 is present (Matsuura et al. 1970 J. Chem. Soc. Perkin Trans. 15: pages 664-670). Physalin A and Physalin B have been isolated and had their structure determined. More than a dozen Physalins have been isolated from Physalis species such as Physalis alkekengi, Physalis angulata, and Physalis lancifolia (see Tomassini et al. U.S. Pat. No. 6,998,394 issued Feb. 14, 2006; and also Mulchandani et al. 1974 BARC-764).

Shown below are the structures of Physalin A, Physalin B, a steroid ring skeleton showing the conventional numbering of a steroid ring, and Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, and Physalin S.

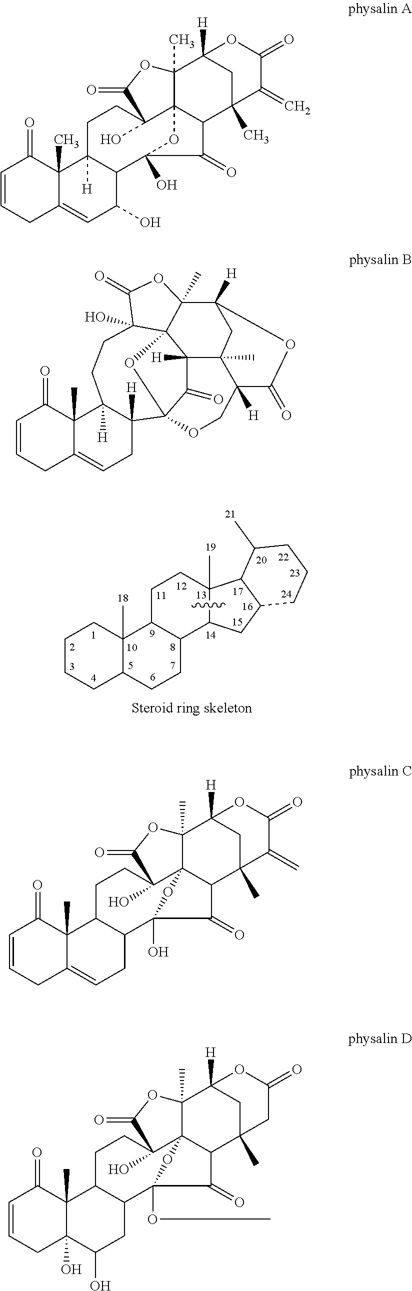

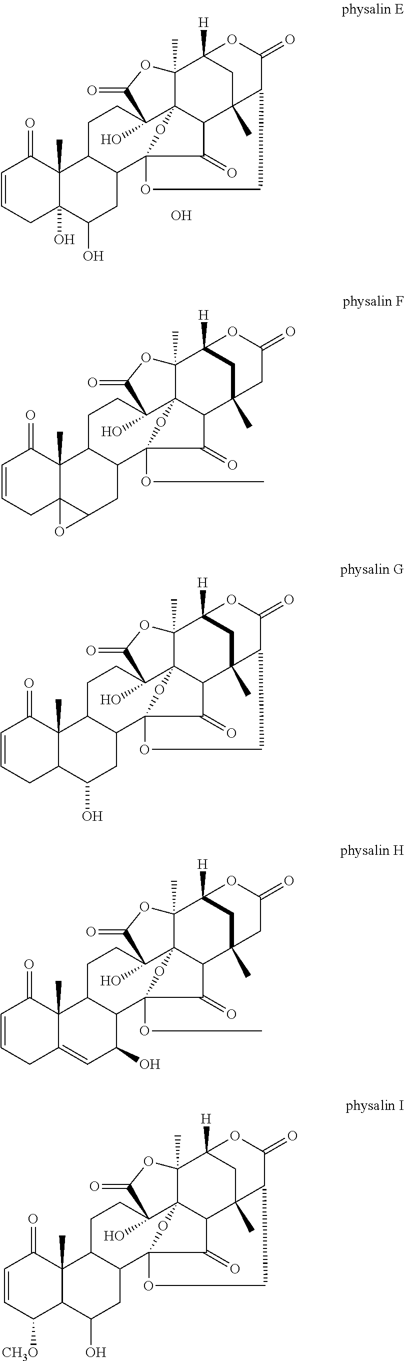

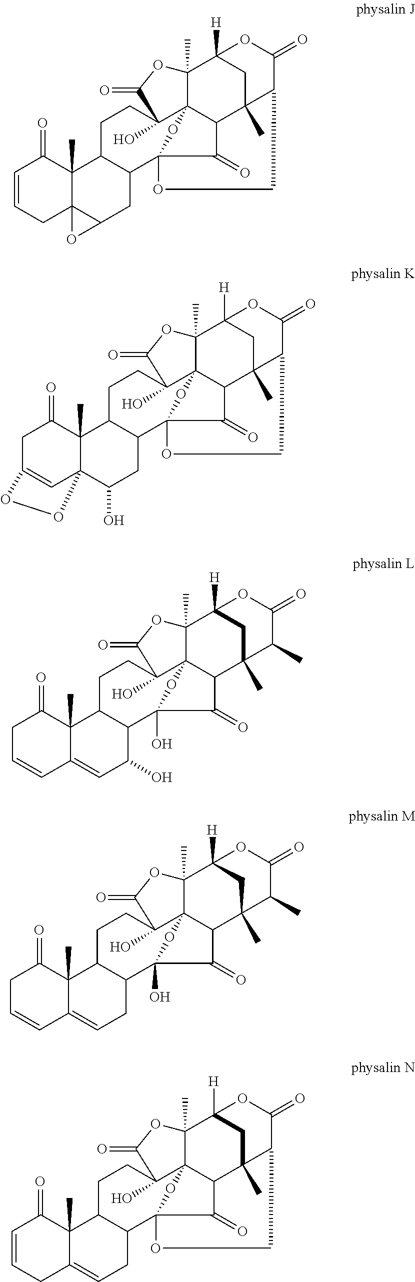

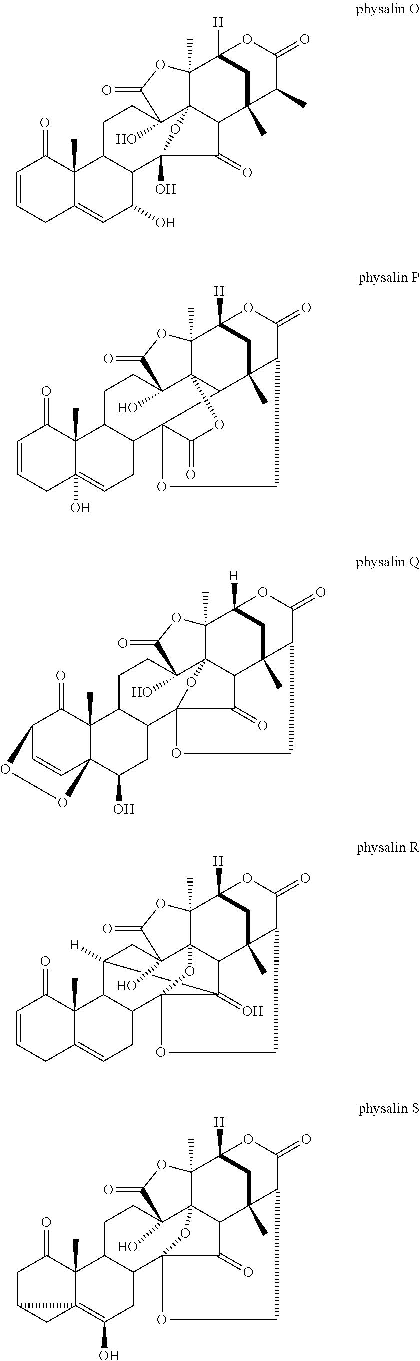

Without being limited by any particular theory or mechanism of action, it is here envisioned that any naturally-occurring or synthetic physalin having a structure containing 13,14-seco-16,24-cyclo-steroidal ring skeleton is effective for inducing neurogenesis in neural stem cells, neural progenitors cells, and differentiated neural cells such as neurons, astrocytes, and oligodentrocytes. Accordingly the term “physalin” as used herein refers to any of these structures (e.g., Physalin A-S), any physalins not yet characterized, including any equivalent structures having modifications, derivatives of physalins, whether the physalins and modifications and derivatives are naturally-occurring, synthetic, or semi-synthetic.

### Preparation of Physalin Extracts and Physalins

It will be appreciated by one of ordinary skill in the art, that the physalin extracts and physalin molecules (e.g., Physalin A and Physalin D) that are embodiments of this invention can be obtained from an available source known to synthesize this class of molecule. These include but are not limited to molecules isolated from natural sources (e.g., Physalis angulata), produced recombinantly or produced synthetically, e.g., by solid phase procedures or organic chemistry methods. In accordance with the present invention, polynucleotide sequences which encode physalins may be used in recombinant DNA molecules that direct the expression of the physalin biosynthetic genes of this invention in appropriate host cells. In order to express a biologically active physalin, the nucleotide sequence encoding the physalins or their functional equivalent, is inserted into an appropriate expression vector, i.e., a vector which contains the necessary elements for the transcription and translation of the inserted coding sequence. Methods which are well known to those skilled in the art can be used to construct expression vectors containing a physalin-encoding sequence and appropriate transcriptional or translational controls. These methods include in vitro recombinant DNA techniques, synthetic techniques and in vivo recombination or genetic recombination. The introduction of deletions, additions, or substitutions can be achieved using any known technique in the art e.g., using PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. A variety of expression vector/host systems may be utilized to contain and express a physalin-encoding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems.

Alternatively, the physalin substances of the present invention are produced using chemical methods to synthesize a physalin, whole or in part. For example, synthesis can be performed using various solid-phase techniques (Roberge et al., Science 269:202, 1995) and automated synthesis may be achieved, for example, using the 431A peptide synthesizer (available from Applied. Biosystems of Foster City, Calif.) in accordance with the instructions provided by the manufacturer. In various embodiments, the physalins are synthesized from or using a steroid such as a withanolide, or a steroid obtain from a Solanaceae genus.

Methods for isolating and preparing physalins and physalin extracts are shown in Tomassini et al. U.S. Pat. No. 6,998,394 issued Feb. 14, 2006, and in Matsura et al. 1970 J. Chem. Soc. (C) 5: pages 664-670, each of which is incorporated by reference herein in its entirety.

### Pharmaceutical Compositions

In one aspect of the present invention, pharmaceutical compositions are provided, wherein these compositions comprise physalins and physalin extracts, and optionally comprise a pharmaceutically acceptable carrier. In certain embodiments, these compositions optionally further comprise one or more additional therapeutic agents. In certain embodiments, the additional therapeutic agent or agents are selected from the group consisting of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), a nerve growth factor (NGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), B vitamins such as biotin, and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In certain embodiments, the physalins are added as a pharmaceutical composition to a cell or a tissue that is used to form a transplant. Methods for preparing transplants are shown for example in international application number PCT/2008/005742 filed May 5, 2008 and international application number PCT/US2010/050288 filed Sep. 24, 2010, each of which is hereby incorporated by reference herein in its entirety.

### Therapeutically Effective Dose

In yet another aspect, according to the methods of inducing or enhancing neurogenesis in cells or tissue by contacting the cells or the tissue with a pharmaceutical composition containing physalin extracts or physalin molecules, as described herein, the method involves using a therapeutically effective dose. Thus, the invention provides methods for the inducing neurogenesis in cells or tissue or treating a neurological disorder or neurological condition involving administering a therapeutically effective amount of a pharmaceutical composition comprising active agents that include physalin extracts or physalins to cells or tissue a subject in need thereof, in such amounts and for such time as is necessary to achieve the desired result.

It will be appreciated that this encompasses administering an inventive pharmaceutical as a therapeutic measure to promote neurogenesis in cells or tissue, or as a prophylactic measure to minimize complications associated with neurological disorder or neurological condition (e.g., minimize neural cell death after an injury, or to stimulate neural stem cells or neural progenitor cells to proliferate and/or differentiate into adult neural cells).

In certain embodiments of the present invention a “therapeutically effective amount” of the pharmaceutical composition is that amount effective for promoting neurogenesis. The compositions, according to the method of the present invention, may be administered using any amount and any route of administration effective for treating the cells or tissue of a subject. Thus, the expression “amount effective for inducing or enhancing neurogenesis”, as used herein, refers to a sufficient amount of composition to promote the proliferation, differentiation, and survival of neural cells in the cells or the tissue. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., extent of neurological disease, history of the condition; age, weight and gender of the patient; diet, time and frequency of administration; drug combinations; reaction sensitivities; and tolerance/response to therapy. Long acting pharmaceutical compositions might be administered several times a day, every day, 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the cells or tissue of the patient, or the patient himself or herself to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, usually mice, rabbits, dogs, or pigs. The animal model is also used to achieve a desirable concentration range and route of administration. While direct application to the subject is performed in certain embodiments as the route of administration, such information from an animal model can then be used to determine useful doses and additional routes for administration in subjects such as subjects such as humans. A therapeutically effective dose refers to that amount of active agent that ameliorates the symptoms or condition (e.g., neurological disorder or neurological condition). Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies are used in formulating a range of dosage for mammalian (e.g., human) use.

A therapeutically effective dose refers to that amount of one or more physalins, e.g., Physalin D, and/or one or more extracts of physalins that induces, enhances or establishes neurogenesis in cells or tissue in a subject. The dosage administered to rodents in the Examples herein was 0.1 mg/kg, 1 mg/kg, or 5 mg/kg. Without being limited by any particular theory or mechanism of action, it is here envisioned that in various embodiments a dosage of one or more physalins and/or extracts of physalins to be administered to other mammalian species including a human weighing about 50 kg to about 100 kg is from about 0.5 mg to about several grams. An effective amount of the one or more physalins and/or extracts of physalins is in various embodiments supplied at a dosage level of from about 0.1 μg (microgram)/kg to about 25 mg/kg of body weight per day. For example, the range is from about 1 μg/kg to 10 mg/kg of body weight per day, or from about 1 μg/kg to about 1 mg/kg of body weight per day.

The effective dose may be administered according to a regimen of, for example once per day or at least twice per day. An effective dose or a unit dose may a divided daily dose. For example, the subject is administered the daily dose in two or more divided doses. In various embodiments, the divided doses contain the same amount of one or more physalins and/or extracts of physalins. Alternatively, the divided doses contain different doses of the one or more physalins and/or extracts of physalins. For example, the subject is administered in a regimen of successive decreasing doses of the amount of one or more physalins and/or extracts of physalins. Alternatively, the subject is administered in a regimen of successive increasing doses of the amount of one or more physalins and/or extracts of physalins (i.e., successive doses that increase the amount of one or more physalins and/or extracts of physalins).

In various embodiments, the daily dosage or the unit dose of the one or more physalins and/or extracts of physalins is within a range of about 0.5 mg to about 10 grams per mammal per day. In various embodiments, the dose is administered in an amount containing at least about: 10 μg, 50 μg, 100 μg, 0.5 mg, 1 mg, 2.5 mg, 5 mg, 10 mg, 15 mg, 25 mg, 50 mg, 100 mg, 150 mg, 250 mg, 325 mg, 400 mg, 500 mg, 750 mg, 1 g (gram), or 2 g per dose.

In various embodiments, a unit dose contains from about 1 μg to about 500 mg of the one or more physalins and/or extracts of physalins or about 500 mg to about 1 gram of the one or more physalins and/or extracts of physalins. In various embodiments, a unit dose contains about 0.1 μg to about 100 μg of one or more physalins and/or extracts of physalins. In various embodiments, the active agent contains about 1 μg to about 10 μg of one or more physalins and/or extracts of physalins.

### Administration of Pharmaceutical Compositions

After formulation with an appropriate pharmaceutically acceptable carrier in a desired dosage, the pharmaceutical compositions of this invention can be administered to humans and other mammals topically for example by powders, ointments, or drops applied directly to epithelial cells or tissues of the subject. Alternative and additional routes are employed such as oral, rectal, parenteral, intracisternal, intravaginal, intraperitoneal, bucal, or nasal, depending on the nature and severity of the condition to be treated. Oral administration of crude aqueous extracts of physalins as tea preparations of Physalis plant material has a successful folk history (Leslie Taylor 2005 “The healing power of rainforest herbs: a guide to understanding and using herbal medicinal” Square One Publishers: 519 pages).

Liquid dosage compositions for ocular administration include suitable salts, buffers and solubilizing agents, preferred diluents such as water, preservatives such as thymosol, and one or more biopolymers or polymers for conditioning the solution, such as polyethylene glycol, hydroxypropylmethylcellulose, sodium hyaluronate, sodium polyacrylate or tamarind gum.

Liquid dosage compositions for oral administration may include, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The oral compositions can also include inert diluents, adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous infections may be treated with aqueous drops, a mist, an emulsion, or a cream. Administration may be therapeutic or it may be prophylactic. Prophylactic formulations may be present or applied to the site of potential wounds or injury, or to sources of wounds. The invention includes neurological devices, opthalmological devices, surgical devices, audiological devices or products which contain disclosed compositions (e.g., gauze bandages or strips), and methods of making or using such devices or products. These devices may be coated with, impregnated with, bonded to or otherwise treated with a disclosed composition.

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage fool's may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Uses of Pharmaceutical Compositions

As discussed above and described in greater detail in the Examples herein, physalin extracts (e.g., aqueous extracts or ethanolic extracts or other organic solvent extracts of a Physalis plant) or physalin molecules (e.g., Physalins A-S, synthetic physalins, semi-synthetic physalins, and derivatives and modifications thereof) are useful as promoters or inducers of neurogenesis in cells or tissues of the body. For example the cells or tissue contain at least one of neural stem cells, neural progenitor cells, or adult neural cells. In certain embodiments, the cells or the tissue include stem cells or progenitor cells. In general, it is believed that these physalin compositions and physalin molecules will be clinically useful in stimulating the healing, proliferation, and function of the cells or the tissue of the subject.

In general, it is shown herein that these physalins and extracts containing physalins are clinically useful in stimulating neurogenesis and healing to cells or tissue that are affected or are at risk for a neurological disorder, neurological disease, or neurological condition.

Without being limited by any particular theory or mechanism of action, it is here envisioned that cell transplants or tissue transplants according to the present invention that contain physalin extracts or physalin molecules are especially useful for facilitating repair, reconstruction and/or regeneration of neural tissue defects in a transplant recipient. The present invention relates to the use of plant extracts containing physalins and physalin molecules to treat neural cells and neural tissue for example in the brain and spinal cord, and to treat damaged tissue of acute and chronic spinal cord injuries, and neurodegenerative diseases of the brain and spinal cord. Compositions, methods and kits are provided for repairing or remediating a neuronal tissue and a spinal cord injury in a subject and for regenerating various neural tissues of the body such as the brain and the spine.

Weiss et al. U.S. Pat. No. 6,497,872 issued Dec. 24, 2002, which is incorporated by reference in its entirety herein, describes methods for the generation of suitable autografts, xenografts, and allografts, describes methods for differentiating stem cells into neural cells (e.g., neurons, astrocytes, and oligodendrocytes), and describes methods for neurotransplantation.

Compositions, methods and kits containing plant extracts containing physalins and physalin molecules to promote and restore neurogenesis in the hippocampal neurogenic regions of the brain and their positive effects in areas related to learning and memory.

An aspect of the invention provides use of a physalin or a physalin extract as a neurologic drug. For example, the physalin includes Physalin D and the physalin extract includes an ethanol extracts containing Physalin D, and aqueous extracts containing Physalin D. In various embodiments, the physalin is at least one selected from: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof.

An aspect of the invention provides use of a starting material include a physalin or a physalin extract for processing new molecules with neurogenic activity. For example, the physalin or physalin extract is obtained from a natural source, such as a plant. In certain embodiments, the natural source is an aerial portion of the plant such as Physalin angulata.

In certain embodiments, the physalin or the physalin extract is fully formulated in a dose for use in pharmaceutical forms with neurogenic activity. In a related embodiment, the physalin or the physalin extract has synergistic activity to maximize a neurogenic effect in an active molecule or active compound. In a related embodiment, the physalin and/or the physalin extract is effective for treatment of neurodegenerative disorders and a lack of memory. In various embodiments, the physalin or physalin is used with growth factors in neurogenic and non-neurogenic areas.

This application claims the benefit of Brazilian patent application serial number PI1 104177-3 A2 filed Jul. 18, 2011 in the Instituto Nacional da Propriedade Industrial, entitled, “Use of physalin D and aqueous and ethanol extracts in the proliferation of neuronal stem cells of the dentate gyrus of adult mice: A new neurogenic molecule” by inventors Milton Nascimento da Silva, Alberto Cardoso Arruda, Mara Silvia Pinheiro Arruda, Gilmara de Nazareth Tavares Bastos, Raque Carvalho Montenegro, and José Luiz Martins do Nascimento, which is hereby incorporated herein by reference in its entirety.

A portion of the examples were presented in a poster in Portuguese entitled “EXTRATO SM2 PROMOVE PROLIFERAçÃO DE CÉLULAS BRDU-POSITIVAS NA CAMADA SUBGRANULAR DO GIRO DENTEADO DE CAMUNDONGOS ADULTOS” by Nascimento, M. V. L.; Magalhãdes, R. C.; Santos I. V. F.; Farias, L. H. S.; Silva, E. O.; Bastos, G. N.; and Do Nascimento, J. L. M., which was presented at the 23rd ISN-ESN Biennial Meeting (ISN—The International Society for Neurochemistry); Aug. 28, 2011-Sep. 1, 2011 in Athens Greece.

A skilled person will recognize that many suitable variations of the methods may be substituted for or used in addition to those described above and in the claims. It should be understood that the implementation of other variations and modifications of the embodiments of the invention and its various aspects will be apparent to one skilled in the art, and that the invention is not limited by the specific embodiments described herein and in the claims. Therefore, it is contemplated to cover the present embodiments of the invention and any and all modifications, variations, or equivalents that fall within the true spirit and scope of the basic underlying principles disclosed and claimed herein.

### EXAMPLES

### Example 1

### Materials and Methods

Adult six to eight week old male BalbC mice were obtained and kept in cages at room temperature, allowed access to food and water ad libidum, and subjected to 12-hour light/darkness cycles. All procedures were conducted in accordance with the guide for laboratory animal care of the Institute of Biological Sciences/Federal University of Pará (ICB/UFPA) Ethics Committee.

For BrdU analysis, animal subjects were administered BrdU (50 mg/kg) and were euthanized, and then were perfused with saline and 4% paraformaldehyde (PFA). The brain tissues were dissected and fixed at 4° C. in 4% PFA for 12 hours, then cryoprotected in 20% sucrose. The brain tissues were sliced at 40 micrometers (microns, μm) in a cryostat. For BrdU immunohistochemistry, the slices were treated with hydrochloric acid (2N) at 37° C. for 20 min, neutralized with sodium borate buffer (0.15 M, pH 8.5) for ten minutes, washed with phosphate buffered saline (PBS) and blocked with 2.5% BSA solution for two hours. The tissue slices were then incubated at room temperature with anti-BrdU primary antibody for 12 hours. The samples were then incubated with secondary antibody according to the specificity of primary antibody used.

### Example 2

### Methods for Obtaining Aqueous and Ethanol Extractions and Purified Physalin D from Physalis angulata

The Physalin D (PD) extraction was performed using the aerial parts of Physalis angulata, which were oven dried at a temperature of 100° C. for two days and then pulverized in a mill, to obtain five kilograms of dried, ground material.

The ethanol extraction was performed as follows. Four kilograms of dried, ground material was soaked for 24 hours with hexane to obtain three extraction which was filtered and the filtered extract (filtrate) was evaporated to remove solvent in a rotary evaporator to obtain a residue. The residue was then extracted during a period of eight hours using ethyl alcohol in seven cycles with a Sohxlet extraction apparatus. The solvent was removed in a rotary evaporator, yielding 70 grams of ethanol extracted material (EE).

The aqueous extraction was performed as follows. An amount (150 grams) of the dried, ground material (oven-dried plant material) was added to 700 milliliters (ml) of water and heated to 100° C. for 10 minutes. A resulting solution (filtered to remove insolubles) was lyophilized, yielding 2.7 grams of lyophilized material of aqueous extract (AE). The AE and EE extracts were stored away from light and humidity.

To obtain purified PD, 50 grams of EE were fractionated by column chromatography using silica gel, and resolved with one liter of each of the following solvent mixtures: hexane/ethyl acetate 90:10 (F1); hexane/ethyl acetate 70:30 (F2); hexane/ethyl acetate 50:50 (F3); ethyl acetate 100% (F4); ethyl acetate/methanol 80:20 (F5) and 100% methanol (F6) which produced, after evaporation of the solvents: 71 mg of F1, 237 mg F2, 950 mg of F3, 6 g of F4, 278 mg F5, and 30 g F6. The PD was obtained from solvent mixture F5 by fractionation using high-performance liquid chromatography with a reverse phase C18 column and a mobile phase consisting of a mixture of water and acetonitrile (75:25) at a flow rate of 4.7 milliliters per minute.

It is envisioned that different aerial plants components, for example, the flower or the fruit, are particularly enriched for the neurogenic physalins.

### Example 3

### BrdU Quantification of Hippocampal Stem Cell Proliferation Using Physalin D

To analyze hippocampal stem cell proliferation and effect of physalins on this proliferation, groups of subjects were administered a dose of 50 mg/kg BrdU intraperitoneally and different doses of PD. Groups of subjects were sacrificed at either 24 hours or 7 days after administration, time points that are equivalent to a period of time for proliferation and for differentiation of hippocampal stem cells, respectively. Bromodeoxyuridine (BrdU) is a synthetic analogue of thymidine which is incorporated into cell DNA during the S-phase of the cell cycle, a measure of cell proliferation. The total number of BrdU-positive cells in the subgranule layer of the hippocampus was quantified. The number of BrdU-positive cells was calculated by measuring the area (ten micrometers corresponds to two cell bodies) between the hilus and the hippocampal granule layer in each cut (Bastos et al. 2008 J. Ethnopharmacol. 118(2): pages 246-251).

A dose response curve of Physalis angulata extracts and PD in the hippocampal neurogenic area was prepared. Subjects were administered a single dose of 50 mg/kg BrdU intraperitoneally and were sacrificed 24 hours after administration of the AE (0.1 mg/kg, 1 mg/kg, or 5 mg/kg). A significant increase in the number of BrdU-positive cells in the dentate gyrus in the AE extracted-treated groups was observed, compared to the control group (control was 92.3±24.2 (n=9), 0.1 mg/kg AE treated subjects was 160.7±21.4 (n=4); 1 mg/kg/kg AE treated subjects 310.5±4.6 (n=4); and 5 mg/kg AE treated subjects was 519±27.7 (n=4), p<0.05). Thus, as quantified by BrdU in cells, it was observed that AE induced neurogenesis in a dose dependent matter.

Without being limited by any particular theory or mechanism of action, it is here envisioned that the AE extract has a proliferative effect on neural stem cells in the dentate gyrus of the hippocampus of the adult mammalian brain.

### Example 4

### Quantification of Hippocampal Stem Cell Proliferation Using Physalin D

A qualitative analysis of BrdU-labeled cells in the dentate gyrus of adult mice was also performed using fluorescence microscopy. Data show the formation and presence of neural cell clusters in the dentate gyrus from subjects that were administered a 0.1 mg/kg dose of AE of Physalis angulata. The cluster of neural cells (a neurosphere) is one of the main characteristics of the neurogenesis process. In the group administered 5 mg/kg of AE of Physalis angulata, BrdU-positive cells appeared to be distributed throughout the hippocampal dentate gyrus and the presence of cell clusters was also observed using fluorescence microscopy. Sporadic BrdU immunostaining was observed in cells from the control subjects administered 10 ng/ml EGF and FGF-2 only, however the immunostaining observed was from a much smaller number of BrdU-positive cells than the cells from subject administered the AE of Physalis angulata.

To further verify the qualitative data obtained using fluorescence microscopy of BrdU, cells were imaged using a Hoechst fluorescent dye that stains and marks nucleic acids in the cell nucleus. Cells were imaged with Hoechst dye to determine whether there was an overlap of images of labeled and formed cells in the dentate gyrus of the various control and experimental groups. Double marking or staining of cells with BrdU and Hoechst dyes indicated presence of new neural cells in the hippocampus.

It was observed that the extracts of Physalis angulata increased the number of BrdU-positive cells in the hippocampal dentate gyrus, and most importantly that the BrdU-positive cells were located primarily in the subgranular zone (SGZ), which is the dentate gyrus region located between the hilus and granule cell layer. The SGZ corresponds to the proliferation site of neural stem cells that divide and migrate to the granule cell layer. Data herein quantified the number of BrdU-positive cells in all sub-regions of the hippocampus dentate gyrus (granule subzone, granule cell layer, molecular, and hilus) and compared the proliferative capacity of the extracts on the other regions of the hippocampus.

The data show that extracts of Physalis angulata or the purified physalins act in the neurogenic niche of the hippocampal dentate gyrus. Ectopic proliferation or the presence of cells outside the granule sub-layer was not observed. Ectopic proliferation is generally considered an undesirable or even a pathological feature, and was not observed in examples herein.

### Example 5

### AE Extract Increased the BrdU-Positive Cells in the Dentate Gyrus In Vitro

To determine whether a dose of 5 mg/kg AE of Physalis angulata would increase the number of BrdU-positive cells in the dentate gyrus during the differentiation of NSCs, subjects were sacrificed seven days after BrdU and AE administration. It was observed that administration of AE resulted in a significant increase in the number of BrdU-positive cells compared to the control group administered BrdU and saline only. The number of BrdU-positive cells from the control subjects was 98.75±7.2 (n=4) and the number of BrdU-positive cells from the 5 mg/kg AE-treated subjects was 130.66±16.8 (n=4). The AE resulted in a 32% enhancement of BrdU-positive cells in the dentate gyrus.

A qualitative analysis of BrdU-labeled cells in the AE-treated subjects and the control subjects was performed. Data show increased formation of a cluster of BrdU-positive cells in subjects administered 5 mg/kg AE extract compared to the control subjects administered saline. Data show that the aqueous SM2 extract of Physalis angulata promoted an increase in BrdU-positive neural cells in the subgranular zone of the dentate gyrus of adult subjects seven days after administration of bromodeoxyuridine.

### Example 6

### Physalin D Increased the BrdU-Positive Cells in the Dentate Gyrus In Vitro

Examples herein performed in vitro analysis of cells from subject cells administered purified PD substance.

The hippocampi of Wistar rats (post natal days one to five) were dissected and mechanically dissociated. Viable cells were plated in four wells of a multi-well culture dish (NUNC Inc.) at a density of 10⁶ cells/well containing neurobasal medium without serum or growth factor. After 24 hours, the cells were washed and PD was added to the cells (treated group). Control cells (absent PD) were treated with 10 nanograms per milliliter (ng/ml) of each of EGF and FGF-2 only. Cultures were incubated at 37° C. with 5% CO₂ and 95% atmospheric air.

Hippocampus cells were cultured with 10 μM of PD for 24 hours, and the cells were then immunostained for using an anti-nestin antibody. Nestin is an intermediate filament protein expressed in dividing cells during the early stages of development in the central nervous system (CNS), peripheral nervous system (PNS) and in myogenic and other tissues. Without being limited by any particular theory or mechanism of action, it is here envisioned that presence of nestin in hippocampus cells indicates the presence of progenitor stem cells.

It was observed that PD increased the number of nestin-positive cells present in the hippocampus cell culture. Data show that hippocampal stem cells were differentiating into progenitor cells. Thus EE and AE of Physalis angulata and PD have neurogenic activity in vivo and in vitro and are bioactive molecule that promote neurogenesis in the hippocampus and in the hippocampal neurogenic niche.

### Example 7

### Process of Neural Stem Cells and Neural Progenitor Cells Proliferation and Differentiation

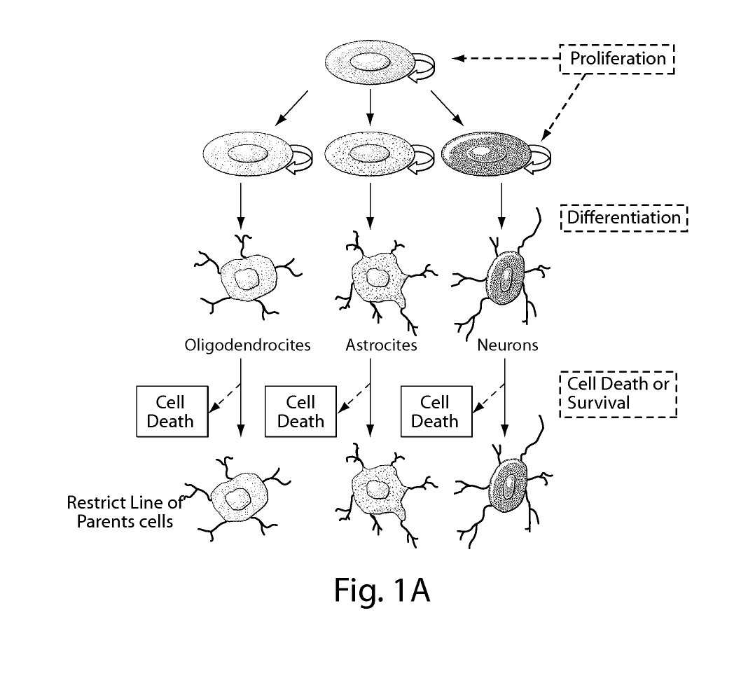

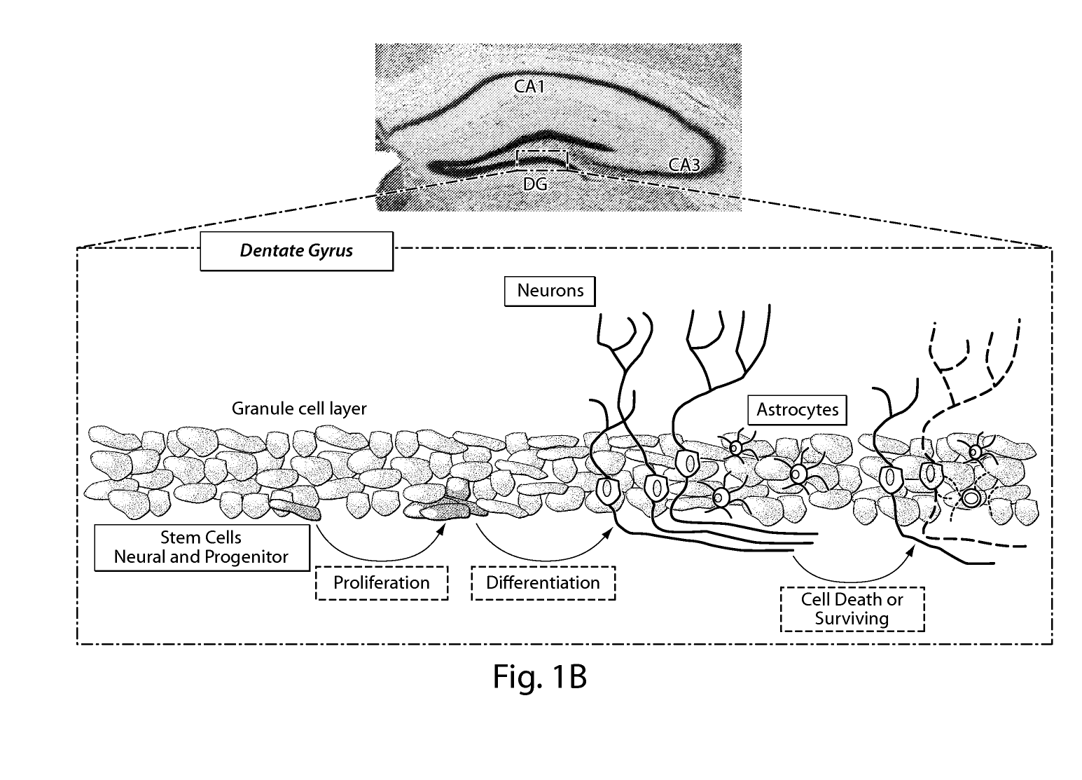

The process of neurogenesis in the dentate gyrus region of the hippocampus involves neural stem cells in the dentate gyrus proliferating into progenitors cells, which differentiate into either oligodentrocytes, astrocytes, or neurons (FIG. 1A). Over a period of time the differentiated mature oligodentrocytes, astrocytes, or neurons either survive or due. FIG. 1 panel B (top) is a photograph of a section of a hippocampus from a BALB/C mouse. The pictures of the hippocampus section shows the hippocampal cornu ammonis (Ammon's horn; CA) 1 area (CA1), the hippocampal CA2 area (CA2) and dentate gyrus (DG). The dentate gyrus consists of three layers of neurons: molecular, granular, and polymorphic. FIG. 1 panel B (bottom) is a drawing of a portion of the granular layer of neurons of the dentate gyrus shown in FIG. 1 panel B (top). The drawing shows the process of stem cells and progenitor cells (left) proliferation into either neural cells or astrocyte cells, which either undergo cellular death or survive.

### Example 8

### SM2 Extract of Physalis angulata Increased the BrdU-Positive Cells in the Dentate Gyrus In Vitro

Aqueous extract SM2 was prepared and isolated from Physalis angulata as described in Examples herein. Using data from examples herein, it was determined that the aqueous extract SM2 was effective to promote increased bromodeoxyuridine staining and increased Hoechst staining in neural cells in the subgranular zone of the dentate gyrus of six to eight week old adult BALB/C subjects. The number of proliferating cells in living tissues was detected by BrdU. Cell number was determined by Hoechst dye which specifically binds to nucleotide sequences having adenine and thymine.

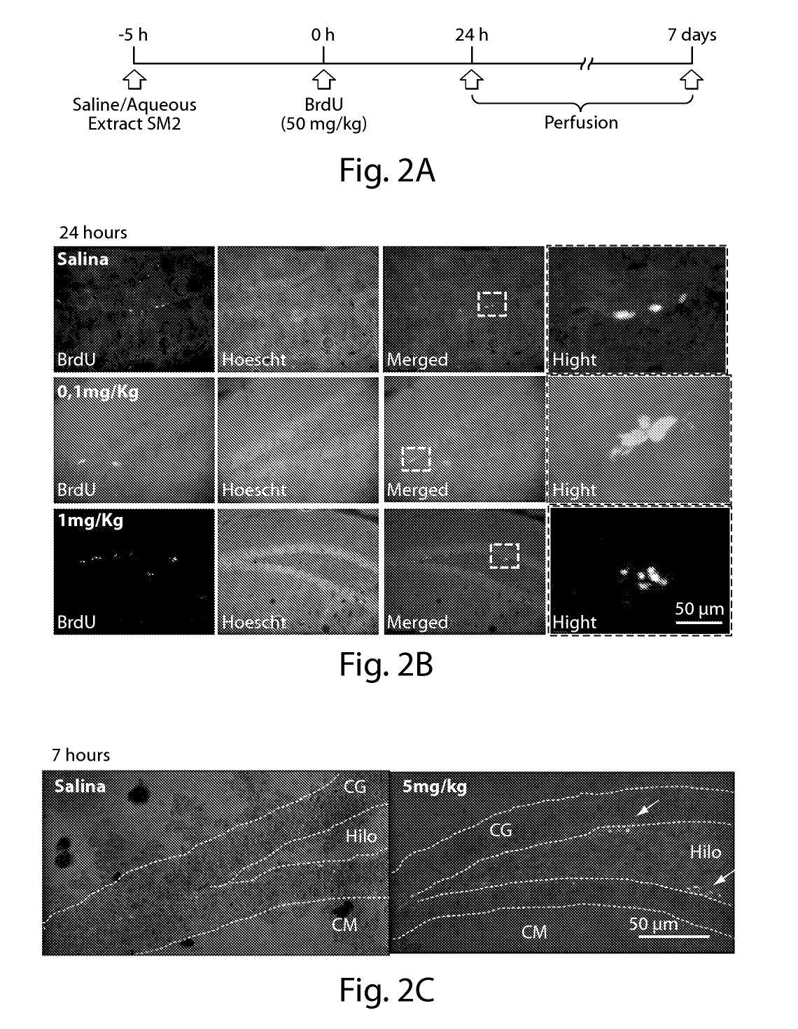

BALB/C subjects were injected with different amounts of the aqueous SM2 extract. Control subjects were injected with saline. Tissues were then administered bromodeoxyuridine (BrdU; 50 mg/kg) five hours later. Subjects were sacrificed and tissues were perfused with saline, stained and visualized twenty-four hours later and seven days later with either BrdU dyes or Hoechst dyes (FIG. 2 panel A).

FIG. 2 panel B is a set of photomicrographs showing BrdU staining (left most column and right most column) and Hoechst staining (second column) of the subgranular zone of the dentate gyrus of BALB/C mice twenty four hours after BrdU administration and injection 0.1 milligram/kilogram weight (mg/kg) of aqueous extract SM2 from Physalis angulata, or 1 mg/kg of aqueous extract SM2 from Physalis angulata. Control subjects were injected with BrdU and saline (FIG. 2 panel B first row). Merged images of the BrdU and Hoechst staining show enhanced BrdU staining and Hoechst staining for each of the subgranular zone of the dentate gyrus of subjects injected with aqueous extract SM2 from Physalis angulata compared to control subjects injected with saline (FIG. 2 panel B third column from left).

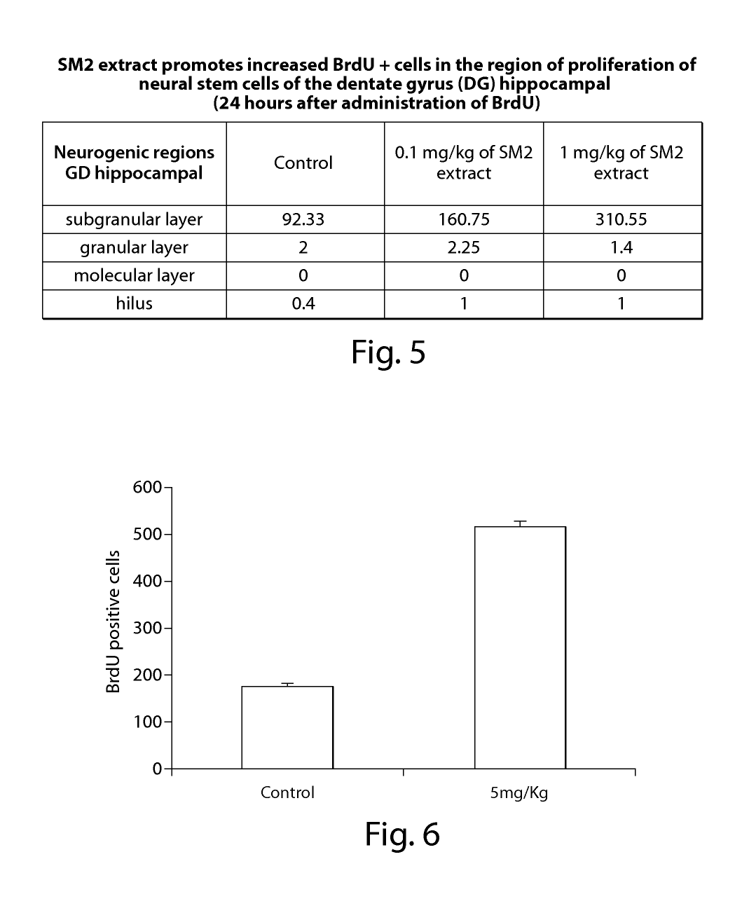

Data show a higher number of BrdU-labeled cells from subjects injected with either 0.1 mg/kg SM2 extract or 1 mg/kg SM2 extract after 24 hours compared to control subjects injected with saline (FIG. 4). Most importantly, data show that aqueous extract SM2 increased in a dose-dependent matter the amount of BrdU in neural cells in the subgranular zone of the dentate gyrus of the hippocampus of subjects compared to control cells (FIG. 5). Cells from subjects injected with 0.1 mg/kg SM2 extract had two-fold more BrdU at 24 hours and cells from subjects injected 1 mg/kg SM2 extract had four-fold more BrdU, than cells from control subjects (FIG. 5)

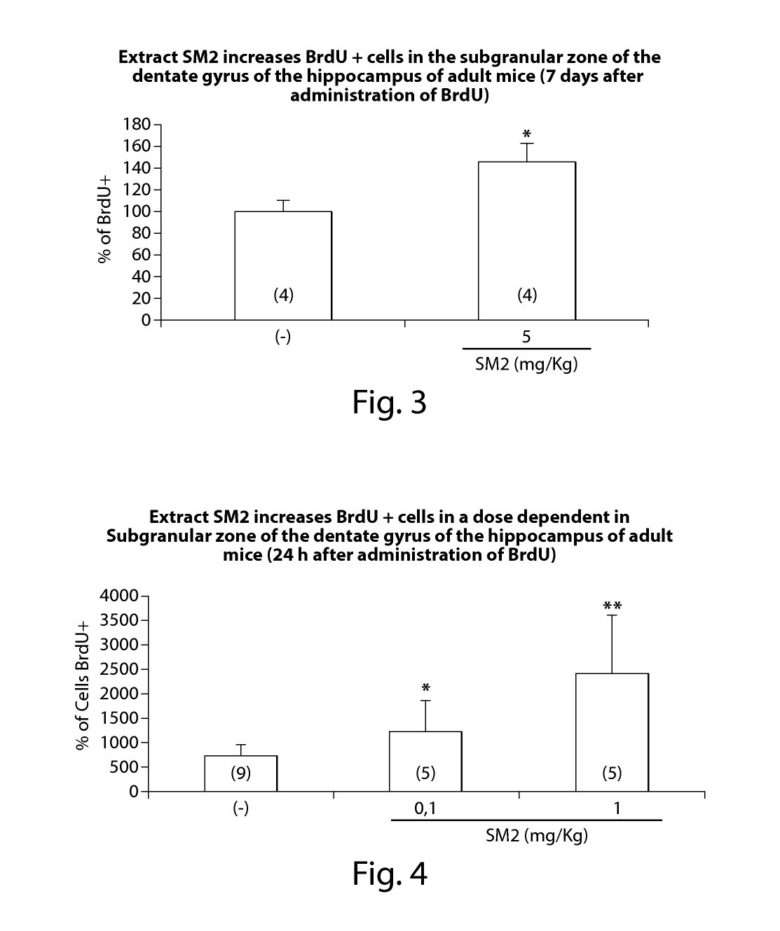

Photomicrographs at seven days after injection and BrdU administration show that aqueous extract SM2 increased BrdU staining in cells in the subgranular zone of the dentate gyrus of subjects compared to BrdU staining in cells from control subjects (FIG. 2 panel C). Quantitative analysis shows that seven days BrdU administration aqueous extract SM2 increased by about 40% the BrdU-labeled neural cells in the subgranular zone of the dentate gyrus of the hippocampus of subjects compared to control cells (FIG. 3). These data show that physalins administered by injection are able to cross the blood-brain barrier and stimulate neurogenesis in the brain.

Without being limited by any particular theory or mechanism of action, it is here envisioned that the SM2 aqueous extract of Physalis angulata contains Physalin D and other physalins that promote neurogenesis in neural cells that form new granule cells in the dentate gyms of the hippocampus. The formation of new neural cells in the hippocampus is a key component of the brain's ability to learn and retain memories, and to counteract affects of neuronal disorders, neuronal diseases, and neuronal conditions. For example, the formation of new neural cells in the hippocampus using physalins is effective to counteract AD, Parkinson's disease, epilepsy, Erb's palsy, Guillain-Barre syndrome, Huntington's disease, and Amyotrophic lateral sclerosis.

### Example 9

### BrdU Quantification of Hippocampal Stem Cell Proliferation Using Physalin D for 90 Days

To further analyze and quantitate hippocampal stem cell proliferation, effect of physalins on this proliferation, and survival of subjects, groups of subjects were administered a dose of 50 mg/kg BrdU intraperitoneally and 5 mg/kg of PD or AE every 7 days for 90 days. The subjects were sacrificed at 90 days from the start of administration. The total number of BrdU-positive cells in the subgranule layer of the hippocampus was quantified. The number of BrdU-positive cells was calculated by measuring the area (ten micrometers corresponds to two cell bodies) between the hilus and the hippocampal granule layer in each cut by the method of Bastos et al. 2008 J. Ethnopharmacol. 118(2): pages 246-251.

The subjects were sacrificed at day 90. It was observed (FIG. 6) that the number of BrdU-positive cells in the dentate gyrus of the brain in AE extract-treated groups was significantly increased, compared to the control group (control was about 180±20, 5 mg/kg AE treated subjects was around 520±20).

The BrdU data quantification demonstrated that AE induced neurogenesis in a dose dependent manner.

Data in this example show that, Physalin D promotes the increase of actively dividing cells in adult hippocampus tissues of the brain and improves cell survival for at least three months, a significant portion of an adult rodent lifespan. Without being limited by any particular theory or mechanism of action, it is here envisioned that the AE extract has a proliferative effect on neural stem cells in the dentate gyrus of the hippocampus of the adult mammalian brain.

### Example 10

### Neurogenesis Induced by Physalins

Mechanical trauma to the central nervous system induces pronounced neuropathology and functional damage. Examples herein determine whether transplants containing physalins or physalin extracts are effective to induce neurogenesis.

A neural injury is created in the spinal cord of adult rats. Animal subjects are analyzed for motor function and sensation in and around the site of the injury. Subjects are administered at the site of the injury neural stem cells containing physalins or physalin extract. Control subjects are administered neural stem cells and saline. Subjects are sacrificed and samples are collected from animal subjects including cerebral spinal fluid and surrounding injured tissue. Samples are stained for cell proliferation and are assayed using appropriate systems, e.g., rat-specific ELISA kits specific for nestin and neural growth factors.

Substantial differences are observed in neural cell number and axonal growth characteristics of subjects implanted with physalins or physalin extracts compared to control animals as much greater cell proliferation staining is observed in the subjects implanted with the physalin or the physalin extract compared to the control subjects. The physalins and physalin extracts are observed to simulate the neurogenesis of cells in the spinal cords of subjects.

What is claimed:

1. A method for inducing neurogenesis in cells or tissue comprising:
administering to the cells or the tissue an agent comprising a physalin, thereby inducing the neurogenesis in the cells and the tissue.

2. The method according to claim 1, wherein administering is performed in vivo in a subject.

3. The method according to claim 1, wherein the subject is at risk for a neurological disease, neurological condition, or a neurological disorder.

4. The method according to claim 3, wherein the neurological disease, neurological condition, or the neurological disorder is selected from the group of: aphasia, encephalomyelitis, adrenoleukodystrophy, agenesis, agnosia, Alexander disease, Alpers' disease, hemiplegia, amyotrophic lateral sclerosis, Alzheimer's disease, and Parkinson's disease.

5. The method according to claim 1, wherein the physalin includes at least one selected from: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof.

6. The method according to claim 1, the method further comprising prior to administering, obtaining the physalin from a natural source.

7. The method according to claim 6, wherein obtaining the physalin comprises extracting the plant with at least one fluid or organic solvent.

8. The method according to claim 6, wherein the natural source is a plant or portion thereof selected from at least one of: a leaf, a stem, and a fruit.

9. The method according to claim 8, wherein the plant is Physalis angulata.

10. The method according to claim 7, wherein the at least one fluid or the solvent is selected from the group consisting of: chloroform, dichloromethane, ethyl acetate, diethyl ether, acetic acid, hexane, toluene, ethanol, acetone, methanol, tetrahydrofuran, dimethyl sulfoxide, acetonitrile, and a combination thereof.

11. The method according to claim 6, wherein obtaining comprises drying or lyophilizing a residue containing the physalin.

12. The method according to claim 1, wherein the physalin is synthetic and prior to administering the method further comprises synthesizing the physalin.

13. The method according to claim 1, wherein the physalin shares at least about 50-90% homology to the following: 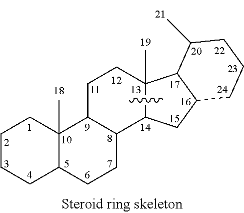
wherein the structure comprises a lactone functionality or an alcohol functionality attached to at least one of carbons 13, 14, 15, 16, 17, 20, 21, 22, 23 and 24.

14. The method according to claim 1, wherein the physalin comprises a 13,14-seco-16,24-cyclo-steroid structure.

15. The method according to claim 1, wherein prior to administering, the method comprises preparing a transplant containing the cells or the tissue of the subject, wherein the cells or the tissue is treated in vitro.

16. The method according to claim 3, further comprising observing a remediation of the neurological disease or the neurological condition.

17. The method according to claim 1, wherein administering the physalin comprises administering an additional therapeutic agent.

18. The method according to claim 17, wherein the therapeutic agent comprises at least one of: a growth factor, an anti-inflammatory agent, a vasopressor agent, a collagenase inhibitor, a steroid, a matrix metalloproteinase inhibitor, an ascorbate, an angiotensin, a calreticulin, a tetracycline, a fibronectin, a collagen, a thrombospondin, an anti-viral, an anti-cancer, an anti-seizure, and an anti-coagulant.

19. The method according to claim 17, wherein the therapeutic agent is selected from at least one of: a drug, a polymer, a protein, a peptide, a carbohydrate, a low molecular weight compound, an oligonucleotide, a polynucleotide, and a genetic material such as DNA or RNA.

20. The method according to claim 1, wherein administering includes at least one selected from the group of: invitreally, subcutaneously, orally, rectally, parenterally, intracisternally, intravaginally, intraperitoneally, bucally, nasally, and intra-cranially.

21. A method for making an agent that induces or enhances neurogenesis in cells or tissue, the method including: extracting a physalin from a natural plant source and administering the physalin to a subject in need of neurogenesis, wherein the physalin induces or enhances the neurogenesis.

22. The method according to claim 21, wherein the natural plant source is a plant or portion thereof selected from at least one of: a leaf, a stem, a flower, a flower bud, and a fruit.

23. The method according to claim 22, wherein the plant is a member of the genus Physalis.

24. The method according to claim 22, wherein extracting the physalin comprises contacting the plant with at least one fluid or organic solvent, for example the fluid is an aqueous fluid.

25. The method according to claim 24, wherein the fluid or the organic solvent is at least one selected from the group consisting of: chloroform, dichloromethane, ethyl acetate, diethyl ether, acetic acid, hexane, toluene, ethanol, acetone, methanol, tetrahydrofuran, dimethyl sulfoxide, acetonitrile, water, and a combination thereof.

26. The method according to claim 21, wherein after extracting, the method further comprises drying or lyophilizing the fluid or the solvent to obtain a residue containing the physalin.

27. A pharmaceutical composition for inducing or enhancing neurogenesis comprising a physalin having a 13,14-seco-16,24-cyclo-steroid structure, and a pharmaceutically acceptable diluent or carrier.

28. The composition according to claim 27 comprising a physalin selected from at least one: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof.

29. The composition according to claim 27, wherein the physalin comprises a structure having a lactone or an alcohol.

30. The composition according to claim 27, wherein the functional group is at one selected from: a (C₁-C₁₈)alkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl.

31. The composition according to claim 27, wherein the functional group is at one selected from: a (C₁-C₁₈)alcohol, a (C₁-C₁₈)nitrile, a (C₁-C₁₈)carbonyl, a (C₁-C₁₈)ester, a (C₁-C₁₈)lactone, a (C₁-C₁₈)ether, and a (C₁-C₁₈)alkoxy.

32. The composition according to claim 27, wherein the functional group is attached to at least one carbon atom in a steroid structure at number: 13, 14, 15, 16, 17, 20, 21, 22, 23 and 24.

33. A pharmaceutical composition for inducing or enhancing neurogenesis, the composition comprising at least one physalin selected from Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof; and a pharmaceutically acceptable diluent or carrier, wherein the physalin is selected for neurogenesis activity and brain physiological tolerance at dosages effective for stimulating brain cell number.

34. A kit for inducing or enhancing neurogenesis comprising:
a physalin including a 13,14-seco-16,24-cyclo-steroid structure and a pharmaceutically acceptable diluent or carrier;
instructions for use in a subject in need of neurogenesis; and,
a container.

35. The kit according to claim 34, wherein the physalin comprises at least one selected from: Physalin A, Physalin B, Physalin C, Physalin D, Physalin E, Physalin F, Physalin G, Physalin H, Physalin I, Physalin J, Physalin K, Physalin L, Physalin M, Physalin N, Physalin O, Physalin P, Physalin Q, Physalin R, Physalin S, and derivatives and modifications thereof.

36. The kit according to claim 34, wherein the physalin is naturally occurring.

37. The kit according to claim 34, wherein the physalin is synthetic or semi-synthetic.

38. The kit according to claim 34, wherein the physalin is from an extract of a plant.

39. The kit according to claim 34, wherein the physalin comprises a structure having a lactone.

40. The kit according to claim 34, wherein the physalin comprises a structure having an alcohol.

41. The kit according to claim 34, wherein a functional group is attached to at least one of carbon atom of the steroid structure.

42. The kit according to claim 41, wherein the functional group is at one selected from: a (C₁-C₁₈)alkyl, a (C₁-C₁₈)alkoxy, a (C₁-C₁₈)heteroalkyl, a (C₆-C₁₀)aryl, a (C₁-C₉)heteroaryl, and a (C₆-C₁₀)aryl(C₁-C₆)alkyl.

43. The kit according to claim 41, wherein the functional group is at one selected from: a (C₁-C₁₈)alcohol, a (C₁-C₁₈)nitrile, a (C₁-C₁₈)carbonyl, a (C₁-C₁₈)ester, a (C₁-C₁₈)lactone, a (C₁-C₁₈)ether, and a (C₁-C₁₈)alkoxy.

44. The kit according to claim 41, wherein the functional group is attached to at least one carbon atom in the steroid structure at number: 13, 14, 15, 16, 17, 20, 21, 22, 23 and 24.
